(19) United States
(12) Patent Application Publication
(10) Pub. No.: US 2026/0069229 A1
(43) Pub. Date: Mar. 12, 2026

(54) METHOD, SYSTEM, AND COMPUTER-READABLE RECORDING MEDIUM FOR DETERMINING ARTHRITIS GRADE BY USING MULTIPLE ARTIFICIAL NEURAL MODELS

(71) Applicant: CONNECTEVE CO., Ltd, Seoul (KR)

(72) Inventor: DaeSeok Song, Incheon (KR)

(21) Appl. No.: 18/946,895

(22) Filed: Nov. 13, 2024

(30) Foreign Application Priority Data
Sep. 12, 2024 (KR) 10-2024-0124529

(51) Int. Cl. A61B 6/50; A61B 6/00; G06N 3/09; G16H 30/40; G16H 50/30

(52) U.S. Cl. CPC A61B 6/505; A61B 6/5217; G06N 3/09; G16H 30/40; G16H 50/30

(57) ABSTRACT

Provided are a method, a system, and a computer-readable recording medium for determining an arthritis grade by using multiple artificial neural models, in which a first model, which corresponds to a CNN-based artificial neural network model, and a third model, which corresponds to a transformer-based artificial neural network model, are trained through training data corresponding to an X-ray image labeled in a first scheme in which the arthritis grade is labeled as being low, a second model, which corresponds to a CNN-based artificial neural network model, and a fourth model, which corresponds to a transformer-based artificial neural network model, are trained through the training data corresponding to the X-ray image labeled in a second scheme in which the arthritis grade is labeled as being high, and the arthritis grade for the X-ray image is determined by using the first model, the second model, the third model, and the fourth model.

### BACKGROUND OF THE INVENTION

### 1. Field of the Invention

The present invention relates to a method, a system, and a computer-readable recording medium for determining an arthritis grade by using multiple artificial neural models, and more particularly, to a method, a system, and a computer-readable recording medium for determining an arthritis grade by using multiple artificial neural models, in which a first model, which corresponds to a CNN-based artificial neural network model, and a third model, which corresponds to a transformer-based artificial neural network model, are trained through training data corresponding to an X-ray image labeled in a first scheme in which the arthritis grade is labeled as being low, a second model, which corresponds to a CNN-based artificial neural network model, and a fourth model, which corresponds to a transformer-based artificial neural network model, are trained through the training data corresponding to the X-ray image labeled in a second scheme in which the arthritis grade is labeled as being high, and the arthritis grade for the X-ray image is determined by using the first model, the second model, the third model, and the fourth model, so that the arthritis grade is diagnosed while implementing a process in which a medical staff makes global/local determination and optimistic/pessimistic determination for the X-ray image in an actual medical site.

### 2. Description of the Related Art

Arthritis is a degenerative disease in which cartilage that absorbs shock is damaged due to aging, excessive exercise, and the like so as to cause articular cartilage to deteriorate. Meanwhile, as the elderly population rapidly increases, the number of patients suffering from the arthritis is rapidly increasing, whereas a process of diagnosing and treating the arthritis takes a long time due to the shortage of medical manpower. In detail, the arthritis may be diagnosed based on complex determination using various data in various perspectives. For example, even when viewing an identical X-ray image, some medical staffs may determine an arthritis grade as being bad, while other medical staffs may determine the arthritis grade as being good.

Alternatively, the medical staff may diagnose the arthritis by globally analyzing an X-ray image of a joint of the patient, or may diagnose the arthritis by locally analyzing a specific part of the X-ray image.

Meanwhile, as a method for rapidly diagnosing arthritis, a method using an artificial intelligence technology has recently been proposed. In detail, the arthritis grade may be automatically diagnosed from the X-ray image of the patient by using a deep learning-based trained artificial intelligence model.

However, a conventional arthritis grade determination technology using the artificial intelligence technology may not reflect both optimistic determination and pessimistic determination for the identical X-ray image, or both global determination and local determination for the X-ray image. As a result, it was not possible to implement a determination process that is similar to a process in which the medical staff determines the arthritis grade in an actual medical site.

### DOCUMENTS OF RELATED ART

### Patent Documents

Korean Patent Registration KR 10-2414601 B1 “Bone density derivation method for hip joint fracture diagnosis based on machine learning and bone density derivation program using the same”

### SUMMARY OF THE INVENTION

An object of the present invention is to provide a method, a system, and a computer-readable recording medium for determining an arthritis grade by using multiple artificial neural models, in which a first model, which corresponds to a CNN-based artificial neural network model, and a third model, which corresponds to a transformer-based artificial neural network model, may be trained through training data corresponding to an X-ray image labeled in a first scheme in which the arthritis grade is labeled as being low, a second model, which corresponds to a CNN-based artificial neural network model, and a fourth model, which corresponds to a transformer-based artificial neural network may be trained through the training data corresponding to the X-ray image labeled in a second scheme in which the arthritis grade is labeled as being high, and the arthritis grade for the X-ray image may be determined by using the first model, the second model, the third model, and the fourth model, so that the arthritis grade may be diagnosed while implementing a process in which a medical staff makes global/local determination and optimistic/pessimistic determination for the X-ray image in an actual medical site.

To achieve the objects described above, there is provided a method for determining an arthritis grade, which is performed by a computing system including at least one processor and at least one memory, the method including: a first determination information derivation step of preprocessing an X-ray image including a joint region, and inputting the preprocessed X-ray image to a first model including an artificial neural network so as to derive first determination information for the arthritis grade; a second determination information derivation step of preprocessing the X-ray image including the joint region, and inputting the preprocessed X-ray image to a second model including an artificial neural network so as to derive second determination information for the arthritis grade; and a final determination step of deriving final determination information for the arthritis grade based on comprehensive determination information including the first determination information and the second determination information, wherein the first model is a deep learning-based artificial neural network model trained by training data labeled in a first scheme, the second model is a deep learning-based artificial neural network model trained by the training data labeled in a second scheme, the first scheme labels labeling information for the arthritis grade to be low as compared with the second scheme for identical training data, which is the X-ray image, and the arthritis grade is numerically expressed in proportion to or in inverse proportion to severity of arthritis.

According to one embodiment of the present invention, the labeling information for the training data before being labeled in the first scheme or the second scheme may be determined as a one-hot vector in which the arthritis grade corresponding to a ground truth value of the training data is determined as 1, and the arthritis grade that does not correspond to the ground truth value is determined as 0 in the arthritis grade divided in levels, the first scheme may be a scheme of inputting an arbitrary number that is lower than 1 in the arthritis grade that is lower than the arthritis grade corresponding to 1 in the labeling information in a form of a one-hot vector, and the second scheme may be a scheme of inputting an arbitrary number that is lower than 1 in the arthritis grade that is higher than the arthritis grade corresponding to 1 in the labeling information of the training data.

According to one embodiment of the present invention, the first model may be trained such that a probability of determining the arthritis grade to be lower than the arthritis grade corresponding to a ground truth value of the training data occurs, and the second model may be trained such that a probability of determining the arthritis grade to be higher than the arthritis grade corresponding to the ground truth value of the training data occurs.

According to one embodiment of the present invention, the first model and the second model may be trained with the training data, which is an identical X-ray image, to which the labeling information is assigned in the first scheme or the second scheme so that only a labeling scheme is different, and the first determination information the and second determination information may include information associated with a numerical value representing that the input preprocessed X-ray image is predicted to correspond to each of a plurality of arthritis grades.

According to one embodiment of the present invention, the first model may include: a plurality of deep learning-based backbone neural network blocks for receiving the preprocessed X-ray image or feature information, which is output from another backbone neural network block of a previous stage, so as to output feature information at a corresponding stage; a plurality of deep learning-based information selection modules for receiving the feature information, which is output from the backbone neural network blocks, so as to output selection information associated with the determination of the arthritis grade from the feature information; and an integrated module for receiving information including a plurality of pieces of selection information, which are output from the information selection modules, so as to output the first determination information for the arthritis grade, and the backbone neural network blocks and the information selection modules may be artificial neural networks that compress data in an identical scheme.

According to one embodiment of the present invention, the method for determining the arthritis grade may further include: a third determination information derivation step of preprocessing the X-ray image including the joint region, and inputting the preprocessed X-ray image to a third model including an artificial neural network so as to derive third determination information for the arthritis grade; and a fourth determination information derivation step of preprocessing the X-ray image including the joint region, and inputting the preprocessed X-ray image to a fourth model including an artificial neural network so as to derive fourth determination information for the arthritis grade, the comprehensive determination information may further include the third determination information and the fourth determination information, and the third model and the fourth model may be artificial neural networks that process or compress data in a different scheme from the first model and the second model.

According to one embodiment of the present invention, the first model and the second model may be convolutional neural network (CNN)- or transformer-based artificial neural network models, and the third model and the fourth model may be: transformer-based artificial neural network models when the first model and the second model are CNN-based artificial neural network models; and CNN-based artificial neural network models when the first model and the second model are transformer-based artificial neural network models.

According to one embodiment of the present invention, the third model may be a deep learning-based artificial neural network model trained by the training data labeled in the first scheme, and the fourth model may be a deep learning-based artificial neural network model trained by the training data labeled in the second scheme.

To achieve the objects described above, there is provided a system for determining an arthritis grade, which includes at least one processor and at least one memory, the system including: a first determination information derivation unit for preprocessing an X-ray image including a joint region, and inputting the preprocessed X-ray image to a first model including an artificial neural network so as to derive first determination information for the arthritis grade; a second determination information derivation unit for preprocessing the X-ray image including the joint region, and inputting the preprocessed X-ray image to a second model including an artificial neural network so as to derive second determination information for the arthritis grade; and a final determination unit for deriving final determination information for the arthritis grade based on comprehensive determination information including the first determination information and the second determination information, wherein the first model is a deep learning-based artificial neural network model trained by training data labeled in a first scheme, the second model is a deep learning-based artificial neural network model trained by the training data labeled in a second scheme, the first scheme labels labeling information for the arthritis grade to be low as compared with the second scheme for identical training data, which is the X-ray image, and the arthritis grade is numerically expressed in proportion to or in inverse proportion to severity of arthritis.

To achieve the objects described above, there is provided a computer-readable recording medium including at least one processor and at least one memory, and configured to perform a method for determining an arthritis grade, wherein the computer-readable recording medium stores instructions for performing steps including: a first determination information derivation step of preprocessing an X-ray image including a joint region, and inputting the preprocessed X-ray image to a first model including an artificial neural network so as to derive first determination information for the arthritis grade; a second determination information derivation step of preprocessing the X-ray image including the joint region, and inputting the preprocessed X-ray image to a second model including an artificial neural network so as to derive second determination information for the arthritis grade; and a final determination step of deriving final determination information for the arthritis grade based on comprehensive determination information including the first determination information and the second determination information, the first model is a deep learning-based artificial neural network model trained by training data labeled in a first scheme, the second model is a deep learning-based artificial neural network model trained by the training data labeled in a second scheme, the first scheme labels labeling information for the arthritis grade to be low as compared with the second scheme for identical training data, which is the X-ray image, and the arthritis grade is numerically expressed in proportion to or in inverse proportion to severity of arthritis.

According to one embodiment of the present invention, an artificial intelligence model may be trained by using training data in which an arthritis grade is labeled as being lower than a ground truth for an arthritis grade of an X-ray image, so that a doctor who optimistically determines the arthritis grade can be implemented.

According to one embodiment of the present invention, an artificial intelligence model may be trained by using training data in which an arthritis grade is labeled as being higher than a ground truth for an arthritis grade of an X-ray image, so that a doctor who pessimistically determines the arthritis grade can be implemented.

According to one embodiment of the present invention, a portion of an X-ray image may be locally analyzed by using a CNN-based artificial intelligence inference model, so that an arthritis grade can be determined.

According to one embodiment of the present invention, an X-ray image may be globally analyzed by using a transformer-based artificial intelligence inference model, so that an arthritis grade can be determined.

According to one embodiment of the present invention, an arthritis grade can be determined from an X-ray image based on feature information derived by an artificial intelligence inference model that locally analyzes the X-ray image in an optimistic perspective, an artificial intelligence inference model that locally analyzes the X-ray image in a pessimistic perspective, an artificial intelligence inference model that globally analyzes the X-ray image in the optimistic perspective, and an artificial intelligence inference model that globally analyzes the X-ray image in the pessimistic perspective.

According to one embodiment of the present invention, even when an arthritis grade of a patient determined by using an artificial intelligence inference model is incorrect, the incorrect arthritis grade can have a result that is similar to an actual arthritis grade of the patient. For example, when the actual arthritis grade of the patient is Level 4, a probability that the artificial intelligence inference model determines the arthritis grade of the patient as Level 3 may be higher than a probability that the artificial intelligence inference model determines the arthritis grade of the patient as Level 0.

### BRIEF DESCRIPTION OF THE DRAWINGS

FIGS. 1A, 1B, and 1C show processes in which a medical staff determines an arthritis grade by using an X-ray image in an actual medical site.

FIG. 2 shows components of a system for determining an arthritis grade according to one embodiment of the present invention.

FIGS. 3A and 3B show training data labeled in a first scheme and a second scheme according to one embodiment of the present invention.

FIGS. 4A and 4B show content associated with training and inference of a first model and a second model according to one embodiment of the present invention.

FIG. 5 shows components of the first model according to one embodiment of the present invention.

FIG. 6 shows content associated with information processing schemes of the first model and the second model according to one embodiment of the present invention.

FIGS. 7A and 7B show content associated with training and inference of a third model and a fourth model according to one embodiment of the present invention.

FIG. 8 shows content associated with information processing schemes of the third model and the fourth model according to one embodiment of the present invention.

FIG. 9 shows content about determination schemes of the first model and the third model according to one embodiment of the present invention.

FIG. 10 shows content about a method for determining an arthritis grade according to one embodiment of the present invention.

FIG. 11 shows a preprocessing process according to one embodiment of the present invention.

FIG. 12 schematically shows internal components of the computing device according to one embodiment of the present invention.

### DETAILED DESCRIPTION OF THE INVENTION

Hereinafter, various embodiments and/or aspects will be described with reference to the drawings. In the following description, numerous specific details are set forth in order to provide a thorough understanding of one or more aspects for the purpose of explanation. However, it will also be appreciated by a person having ordinary skill in the art that such aspect(s) may be carried out without the specific details. The following description and accompanying drawings will be set forth in detail for specific illustrative aspects among one or more aspects. However, the aspects are merely illustrative, some of various ways among principles of the various aspects may be employed, and the descriptions set forth herein are intended to include all the various aspects and equivalents thereof.

In addition, various aspects and features will be presented by a system that may include a plurality of devices, components and/or modules or the like. It will also be understood and appreciated that various systems may include additional devices, components and/or modules or the like, and/or may not include all the devices, components, modules or the like recited with reference to the drawings.

The term “embodiment”, “example”, “aspect”, “exemplification”, or the like as used herein may not be construed in that an aspect or design set forth herein is preferable or advantageous than other aspects or designs. The terms ‘unit’, ‘component’, ‘module’, ‘system’, ‘interface’ or the like used in the following generally refer to a computer-related entity, and may refer to, for example, hardware, software, or a combination of hardware and software.

In addition, the terms “include” and/or “comprise” specify the presence of the corresponding feature and/or component, but do not preclude the possibility of the presence or addition of one or more other features, components or combinations thereof. In addition, the terms including an ordinal number such as first and second may be used to describe various components, however, the components are not limited by the terms. The terms are used only for the purpose of distinguishing one component from another component. For example, the first component may be referred to as the second component without departing from the scope of the present invention, and similarly, the second component may also be referred to as the first component. The term “and/or” includes any one of a plurality of related listed items or a combination thereof.

In addition, in embodiments of the present invention, unless defined otherwise, all terms used herein including technical or scientific terms have the same meaning as commonly understood by those having ordinary skill in the art. Terms such as those defined in generally used dictionaries will be interpreted to have the meaning consistent with the meaning in the context of the related art, and will not be interpreted as an ideal or excessively formal meaning unless expressly defined in the embodiment of the present invention.

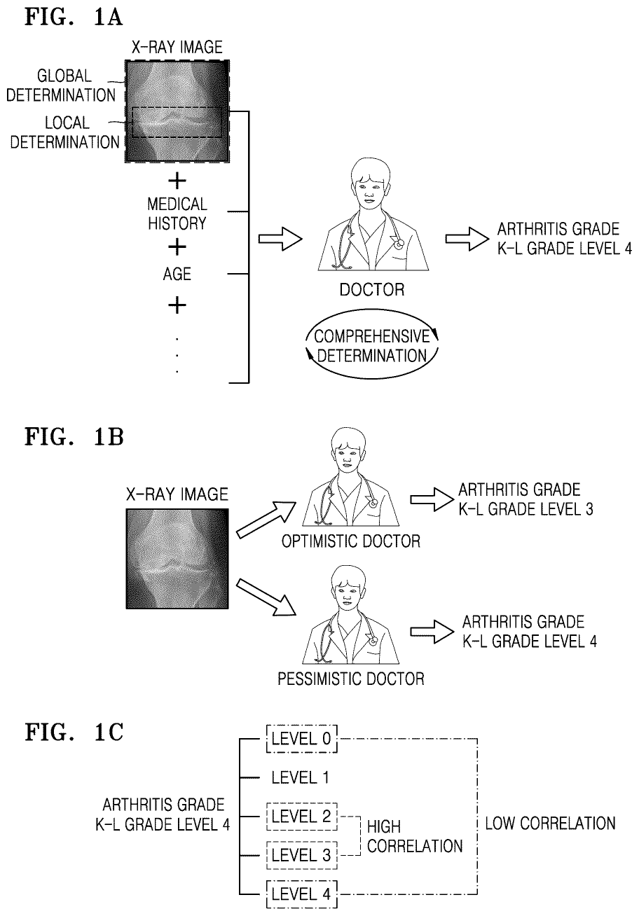

FIGS. 1A, 1B, and 1C show processes in which a medical staff determines an arthritis grade by using an X-ray image in an actual medical site.

As shown in FIG. 1A, a process in which a medical staff determines an arthritis grade of a patient may be performed based on comprehensive determination based on various information. In detail, the medical staff may basically determine the arthritis grade of the patient by analyzing an X-ray image obtained by capturing a joint region (preferably a knee joint) of the patient.

The arthritis grade may be divided and classified into K-L grades in levels. In detail, K-L grades may be divided into Level 0, Level 1, Level 2, Level 3, and Level 4, and the arthritis grade may become worse as the level increases.

For example, Level 0 may correspond to a normal condition with no particular problems in the X-ray image, Level 1 may correspond to a condition in which stenosis of a joint space is ambiguous or an osteophyte may be formed, Level 2 may correspond to a condition in which stenosis of a joint space is possible or an osteophyte is formed for certain, Level 3 may correspond to a condition in which stenosis of a joint space exists, a plurality of moderate osteophytes exist, and subchondral sclerosis and bone deformation may occur, and Level 4 may correspond to a condition in which stenosis of a joint space is severe, an osteophyte is large, and subchondral sclerosis and bone deformation are severe.

The medical staff may analyze the X-ray image of the patient in various perspectives to diagnose the arthritis grade of the patient as one of the K-L grades of Level 0, Level 1, Level 2, Level 3, and Level 4.

In detail, the medical staff may determine the arthritis grade of the patient by analyzing a joint interval, a cartilage condition, an osteophyte, a bone bending condition, and the like while globally viewing the X-ray image. Alternatively, the medical staff may determine the arthritis grade of the patient by focusing on a specific portion (e.g., an osteophyte, a cyst, and a joint interval in each of an inner side and an outer side of a joint) of the X-ray image.

As described above, even with an identical X-ray image, the arthritis grade of the patient may be determined differently depending on how the medical staff views (diagnoses). Alternatively, even with the identical X-ray image, the arthritis grade of the patient may be determined differently depending on the medical staff.

In addition, the medical staff may determine the arthritis grade of the patient by further considering additional information (e.g., an age, a medical history, and a family history of the patient) and the like associated with the patient as well as the X-ray image of the patient.

As shown in FIG. 1B, the arthritis grade may be determined differently depending on tendencies of medical staffs. For example, even with the identical X-ray image, a doctor who globally and optimistically diagnoses the arthritis grade of the patient may diagnose the arthritis grade of the patient as being low. Meanwhile, a doctor who globally and pessimistically diagnoses the arthritis grade of the patient may diagnose the arthritis grade of the patient as being high.

For example, for the identical X-ray image, an optimistic doctor may determine the arthritis grade as Level 3, while a pessimistic doctor may determine the arthritis grade as Level 4, which is worse than Level 3. In other words, the pessimistic doctor may be understood as a doctor who tends to determine that a joint condition is not good as compared with the optimistic doctor.

As described above, when the medical staff analyzes the X-ray image of the patient to determine the arthritis grade in an actual medical site, the arthritis grade may be determined differently depending on an analysis scheme (whether to globally analyze the X-ray image or to locally analyze a specific portion of the X-ray image), a perspective (whether to determine optimistically or pessimistically), and a scheme of interpreting the additional information on the patient (the age, the medical history, the family history, and the like of the patient).

As a result, the arthritis grade determined according to various analysis schemes and perspectives may be comprehensively considered to finally determine the arthritis grade for the patient in the actual medical site, and the present invention may determine the arthritis grade of the patient in a scheme that is similar to the process performed in the actual medical site as described above, so that a system for determining an arthritis grade, which is realistic and highly accurate, may be implemented.

Meanwhile, as shown in FIG. 1C, the arthritis grade may be numerically expressed in proportion to or in inverse proportion to severity of the arthritis. In detail, the arthritis grade may be divided into K-L grades from Level 0 to Level 4, and the arthritis may be understood as being severe as a numerical value increases. In addition, the arthritis grade may be configured such that levels having numerical values that are close to each other have relatively high correlations.

For example, Level 0 representing a best condition and Level 4 representing a worst condition may have the least correlation with each other. In other words, when the X-ray image of the patient corresponding to Level 0 is given to the medical staff, a probability that the medical staff may determine the arthritis grade as Level 1 may be higher than a probability that the medical staff may determine the arthritis grade as Level 4.

Meanwhile, (when compared to a correlation between Level 0 and Level 4), Level 2 and Level 3 may have a high correlation with each other. In other words, when the X-ray image of the patient corresponding to Level 3 is given to the medical staff, a probability that the medical staff may determine the arthritis grade as Level 2 or Level 4 (which is numerically close to Level 3) may be higher than a probability that the medical staff may determine the arthritis grade as Level 0 (which is numerically far from Level 3).

As described above, a correlation between levels may become high as a difference in numerical values (levels) of the arthritis grade classified into the K-L grades decreases, and the correlation between levels may become low as the difference in the numerical values (levels) of the arthritis grade increases.

The system for determining the arthritis grade according to the present invention, which will be described below, may determine the arthritis grade of the patient while reflecting characteristics of the diagnosis process performed in the actual medical site as described above in FIG. 1 and characteristics of the arthritis grade.

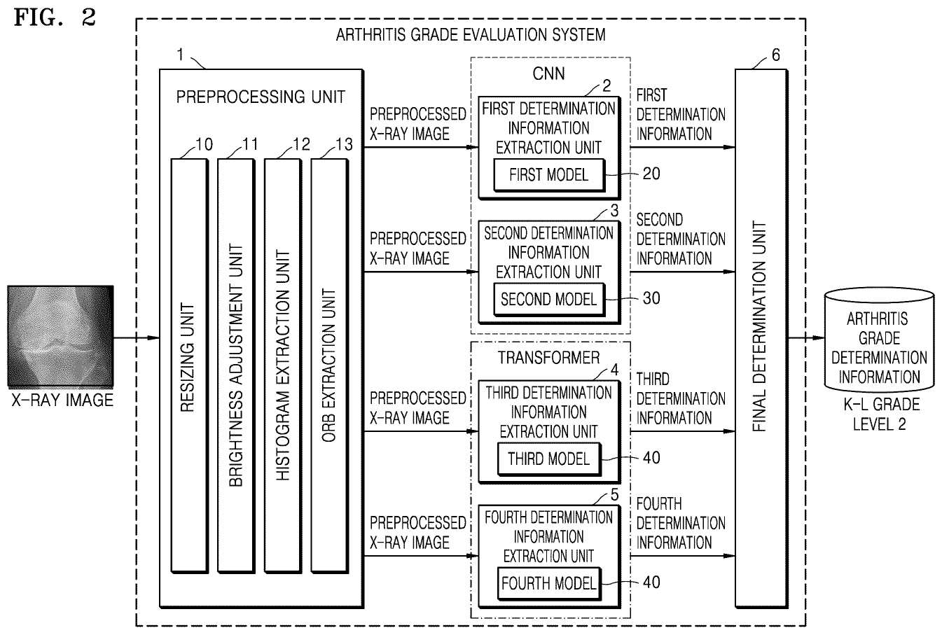

FIG. 2 shows components of a system for determining an arthritis grade according to one embodiment of the present invention.

As shown in FIG. 2, a method for determining an arthritis grade, which is performed by a computing system including at least one processor and at least one memory, may include: a first determination information derivation step of preprocessing an X-ray image including a joint region, and inputting the preprocessed X-ray image to a first model 20 including an artificial neural network so as to derive first determination information for the arthritis grade; a second determination information derivation step of preprocessing the X-ray image including the joint region, and inputting the preprocessed X-ray image to a second model 30 including an artificial neural network so as to derive second determination information for the arthritis grade; and a final determination step of deriving final determination information for the arthritis grade based on comprehensive determination information including the first determination information and the second determination information, wherein the first model 20 may be a deep learning-based artificial neural network model trained by training data labeled in a first scheme, the second model 30 may be a deep learning-based artificial neural network model trained by the training data labeled in a second scheme, the first scheme may label labeling information for the arthritis grade to be low as compared with the second scheme for identical training data, which is the X-ray image, and the arthritis grade may be numerically expressed in proportion to or in inverse proportion to severity of arthritis.

In detail, the system for determining the arthritis grade according to the present invention may receive the X-ray image including the joint region so as to derive the determination information for the arthritis grade of the X-ray image, and the arthritis grade may be expressed as a numerical value in levels, which is divided into the K-L grades.

Preferably, the determination information may be derived as one of the K-L grades of Level 0 to Level 4. For example, the determination information may be derived such that the X-ray image corresponds to the K-L grade of Level 2.

Alternatively, the determination information may be derived as a probability for each of the K-L grades of Level 0 to Level 4. For example, the determination information may be derived as a probability that the X-ray image corresponds to each of the K-L grades of Level 0, Level 1, Level 2, Level 3, and Level 4.

The system for determining the arthritis grade may include a preprocessing unit 1 for preprocessing the X-ray image, and the preprocessing unit 1 may include a resizing unit 10 for resizing a size of the X-ray image, a brightness adjustment unit 11 for adjusting brightness of the X-ray image, a histogram extraction unit 12 for extracting a histogram from the X-ray image, and an oriented FAST and rotated BRIEF (ORB) extraction unit 13 for extracting an ORB from the X-ray image.

The preprocessing unit 1 including the components described above may process the X-ray image in a form that may be input to each of artificial intelligence inference models. For example, the preprocessing unit 1 may adjust the size of the X-ray image to an image size that may be analyzed by each of the first model 20, the second model 30, a third model 40, and a fourth model 50, or may extract the histogram and the ORB required for determining the arthritis grade.

As described above, the preprocessing unit 1 may process the X-ray image in a form optimized for each of the first model 20, the second model 30, the third model 40, and the fourth model 50.

The X-ray image preprocessed by the preprocessing unit 1 may be input to each of the first model 20, the second model 30, the third model 40, and the fourth model 50. In detail, the first model 20, the second model 30, the third model 40, and the fourth model 50 may be deep learning-based trained artificial intelligence inference models.

Preferably, the first model 20 and the second model 30 may be artificial intelligence inference models having the same properties while having different properties from the third model 40 and the fourth model 50. For example, as shown in FIG. 2, the first model 20 and the second model 30 may be convolutional neural network (CNN)-based artificial intelligence inference models, and the third model 40 and the fourth model 50 may be transformer-based artificial intelligence inference models.

In addition, the first model 20 and the second model 30 may be artificial intelligence inference models trained with training data labeled in different schemes, and the third model 40 and the fourth model 50 may be artificial intelligence inference models trained with the training data labeled in different schemes.

In detail, the first model 20 and the third model 40 may be artificial intelligence inference models trained with the training data labeled in a first scheme, and the second model 30 and the fourth model 50 may be artificial intelligence inference models trained with the training data labeled in a second scheme.

According to one embodiment of the present invention, the same artificial intelligence inference model may be trained with the training data labeled in the first scheme or the second scheme, in which the first scheme and the second scheme are different from each other, so that an artificial intelligence inference model that optimistically determines the arthritis grade and an artificial intelligence inference model that pessimistically determines the arthritis grade may be implemented. Detailed descriptions thereof will be given below.

As described above, the system for determining the arthritis grade may include a plurality (preferably 4) of artificial intelligence inference models trained with the training data labeled in different schemes (the first scheme or the second scheme) and having different properties (the CNN or the transformer).

Meanwhile, each of the artificial intelligence inference models may derive the determination information for the arthritis grade. In detail, the first model 20 may receive the preprocessed X-ray image so as to derive first determination information for the arthritis grade, the second model 30 may receive the preprocessed X-ray image so as to derive the second determination information for the arthritis grade, the third model 40 may receive the preprocessed X-ray image so as to derive third determination information for the arthritis grade, and the fourth model 50 may receive the preprocessed X-ray image so as to derive fourth determination information for the arthritis grade.

In addition, each of the first determination information, the second determination information, the third determination information, and the fourth determination information may be derived as a one of the K-L grades of Level 0 to Level 4 or as a probability for each of the levels.

Meanwhile, according to one embodiment of the present invention, the system for determining the arthritis grade may be implemented in the form including at least two of the first model 20, the second model 30, the third model 40, and the fourth model 50. For example, the system for determining the arthritis grade may be implemented in the form including only the first model 20 and the second model 30.

A final determination unit 6 may derive final determination information for the arthritis grade based on comprehensive determination information including the first determination information, the second determination information, the third determination information, and the fourth determination information derived from the first model 20, the second model 30, the third model 40, and the fourth model 50, respectively.

For example, the final determination unit 6 may derive the final determination information based on the first determination information, the second determination information, the third determination information, and the fourth determination information by using a random forest algorithm.

As a result, the system for determining the arthritis grade may derive information on which the level of the K-L grade the input X-ray image corresponds to.

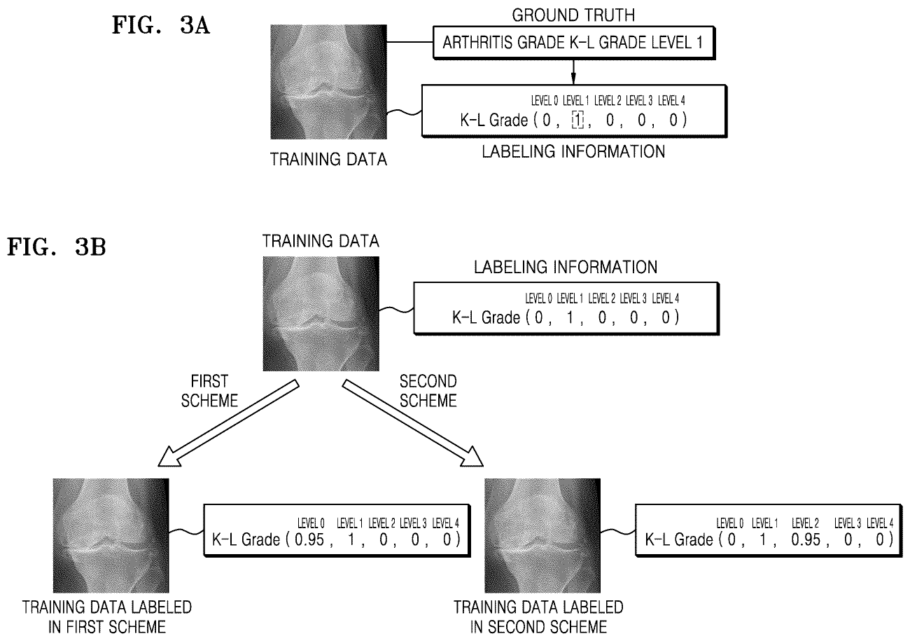

FIGS. 3A and 3B show training data labeled in a first scheme and a second scheme according to one embodiment of the present invention.

As shown in FIG. 3, the labeling information for the training data before being labeled in the first scheme or the second scheme may be determined as a one-hot vector in which the arthritis grade corresponding to a ground truth value of the training data is determined as 1, and the arthritis grade that does not correspond to the ground truth value is determined as 0 in the arthritis grade divided in levels, the first scheme may be a scheme of inputting an arbitrary number that is lower than 1 in the arthritis grade that is lower than the arthritis grade corresponding to 1 in the labeling information in the form of a one-hot vector, and the second scheme may be a scheme of inputting an arbitrary number that is lower than 1 in the arthritis grade that is higher than the arthritis grade corresponding to 1 in the labeling information of the training data.

Hereinafter, the training data may preferably be an X-ray image.

As shown in FIG. 3A, the labeling information for the training data before being labeled in the first scheme or the second scheme may be determined according to the ground truth value of the training data. In detail, in FIG. 3A, when the training data having the K-L grade of Level 1 as the ground truth value exists, the labeling information of the training data may be determined such that a numerical value for Level 1 corresponding to the ground truth value is 1, and numerical values for Level 0, Level 2, Level 3, and Level 4 that do not correspond to the ground truth value are 0.

In other words, in FIG. 3A, the training data may be an actual X-ray image of the patient having the K-L grade of Level 1, and the labeling information may be understood as being labeled such that a probability that the training data corresponds to each of Level 0, Level 2, Level 3, and Level 4 is proportional to 0, and a probability that the training data corresponds to Level 1 is proportional to 1.

As described above, the labeling information may be information in the form of a one-hot vector in which the arthritis grade corresponding to the ground truth value is determined as 1, and the arthritis grade that does not correspond to the ground truth value is determined as 0 in the arthritis grade divided in levels.

Meanwhile, according to one embodiment of the present invention, as described above, the artificial intelligence inference model (the first model 20, the second model 30, the third model 40, and the fourth model 50) may be trained with the training data after the labeling information is artificially processed in the first scheme or the second scheme without training the artificial intelligence inference model with the training data in which the labeling information is labeled.

As shown in FIG. 3B, the first scheme may be a scheme of artificially processing the labeling information to decrease the arthritis grade. For example, in FIG. 3B, the first scheme may be a scheme of arbitrarily modifying the numerical value of the arthritis grade (Level 0) that is lower than the arthritis grade in which the numerical value is 1 in the labeling information (Level 1) from 0 to an arbitrary number (0.95). Meanwhile, in the first scheme, the numerical values of the arthritis grade (Level 2, Level 3, and Level 4) that are higher than the arthritis grade in which the numerical value is 1 (Level 1) may not be modified from 0.

Preferably, the first scheme may be a scheme of modifying the numerical value of the level of the arthritis grade, which is closest to and lower than the level of the arthritis grade in which the numerical value is determined as 1 in the labeling information according to the ground truth value of the training data, to a numerical value that is lower than 1.

In other words, in the first scheme, when the level of the arthritis grade determined as 1 in the labeling information is Level 2, Level 1, which is closest to and smaller than Level 2, may be modified to 0.95, which is an arbitrary number that is smaller than 1, and Level 0, which is closest to and lower than Level 2, may be determined as 0 without being modified.

In other words, in FIG. 3B, the training data may be an actual X-ray image of the patient having the K-L grade of Level 1, and the labeling information of the training data labeled in the first scheme may be understood as being labeled such that a probability that the training data corresponds to Level 1 is proportional to 1, a probability that the training data corresponds to Level 0, which is lower than Level 1, is proportional to 0.95, and a probability that the training data corresponds to each of Level 2, Level 3, and Level 4 is proportional to 0.

As shown in FIG. 3B, the second scheme may be a scheme of artificially processing the labeling information to increase the arthritis grade. For example, in FIG. 3B, the second scheme may be a scheme of arbitrarily modifying the numerical value of the arthritis grade (Level 2) that is higher than the arthritis grade in which the numerical value is 1 in the labeling information (Level 1) from 0 to an arbitrary number (0.95). Meanwhile, in the second scheme, the numerical value of the arthritis grade (Level 0) that is lower than the arthritis grade in which the numerical value is 1 (Level 1) may not be modified from 0.

Preferably, the second scheme may be a scheme of modifying the numerical value of the level of the arthritis grade, which is closest to and higher than the level of the arthritis grade in which the numerical value is determined as 1 in the labeling information according to the ground truth value of the training data, to a numerical value that is lower than 1.

In other words, in the second scheme, when the level of the arthritis grade determined as 1 in the labeling information is Level 2, Level 3, which is closest to and higher than Level 2, may be modified to 0.95, which is an arbitrary number that is smaller than 1, and Level 4, which is closest to and higher than Level 2, may be determined as 0 without being modified.

In other words, in FIG. 3B, the training data may be an actual X-ray image of the patient having the K-L grade of Level 1, and the labeling information of the training data labeled in the second scheme may be understood as being labeled such that a probability that the training data corresponds to Level 1 is proportional to 1, a probability that the training data corresponds to Level 2, which is higher than Level 1, is proportional to 0.95, and a probability that the training data corresponds to each of Level 0, Level 3, and Level 4 is proportional to 0.

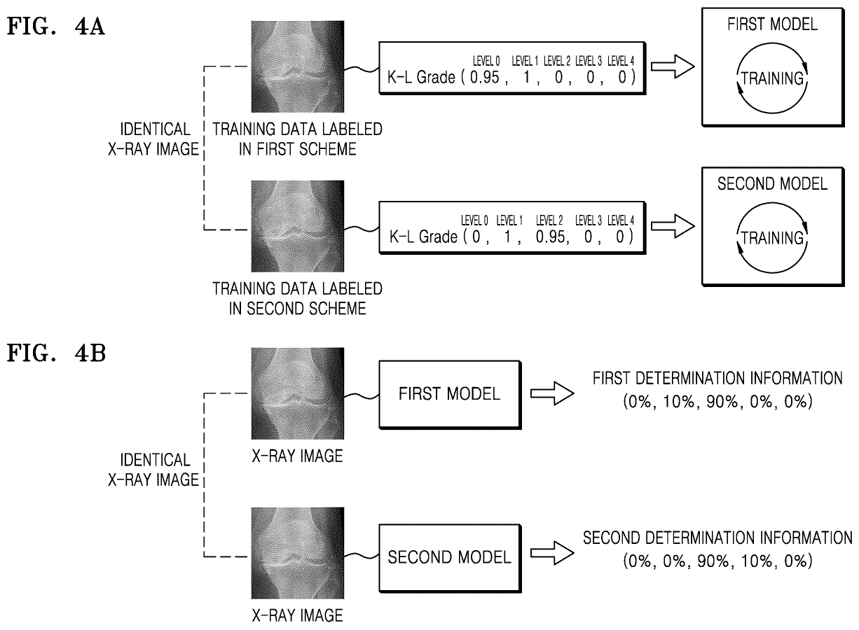

FIGS. 4A and 4B show content associated with training and inference of a first model 20 and a second model 30 according to one embodiment of the present invention.

As shown in FIG. 4, the first model 20 may be trained such that a probability of determining the arthritis grade to be lower than the arthritis grade corresponding to a ground truth value of the training data occurs, and the second model 30 may be trained such that a probability of determining the arthritis grade to be higher than the arthritis grade corresponding to the ground truth value of the training data occurs.

In addition, the first model 20 and the second model 30 may be trained with the training data, which is an identical X-ray image, to which the labeling information is assigned in the first scheme or the second scheme so that only a labeling scheme is different, and the first determination information and the second determination information may include information associated with a numerical value representing that the input preprocessed X-ray image is predicted to correspond to each of a plurality of arthritis grades.

As shown in FIG. 4A, the first model 20 may be trained with the training data labeled in the first scheme (where the labeling information for the arthritis grade is labeled low as compared with the second scheme), and conversely, the second model 30 may be trained with the training data labeled in the second scheme (where the labeling information for the arthritis grade is labeled high as compared with the first scheme).

In detail, the first model 20 and the second model 30 may be understood as the same artificial intelligence inference models while being trained with different training data for training the artificial intelligence inference model. Preferably, the first model 20 and the second model 30 may be understood as being trained with the training data in which only the labeling information is labeled differently for the identical X-ray image.

As described above, the first model 20 and the second model 30 may be the same artificial intelligence inference models in which internal structures and data processing schemes may be the same. In detail, when the first model 20 is a CNN-based artificial intelligence inference model, the second model 30 may also be a CNN-based artificial intelligence inference model. Meanwhile, when the first model 20 is a transformer-based artificial intelligence inference model, the second model 30 may also be a transformer-based artificial intelligence inference model.

In addition, the internal structures of the first model 20 and the second model 30 may be the same, and detailed descriptions thereof will be given below.

As shown in FIG. 4B, the first model 20 may have a high probability of determining the arthritis grade to be low in the X-ray image (as compared with the second model 30), and conversely, the second model 30 may have a high probability of determining the arthritis grade to be high in the X-ray image (as compared with the first model 20).

In detail, the X-ray image may be input to the first model 20 that has been trained so as to output the first determination information, and the X-ray image may be input to the second model 30 that has been trained so as to output the second determination information. In this case, a probability that the arthritis grade in the second determination information is lower than the arthritis grade in the first determination information may be high.

For example, in FIG. 4B, for the identical X-ray image with the K-L grade of Level 2, the first model 20 may determine with a predetermined probability that the X-ray image is at Level 1, which is lower than Level 2.

Preferably, the first model 20 may determine the arthritis grade as a lower level than an actual arthritis grade of the X-ray image. However, even when the arthritis grade is determined as a lower level than the actual arthritis grade, a probability of determining the arthritis grade as “an arthritis grade that is closest to (with the smallest difference) and lower than the actual arthritis grade” may be higher than a probability of determining the arthritis grade as “an arthritis grade that is not closest to and lower than the actual arthritis grade” or “an arthritis grade that is higher than the actual arthritis grade”.

For example, when the X-ray image with the arthritis grade of Level 2 is input to the first model 20, even when the first model 20 determines the arthritis grade to be lower than Level 2, a probability of determining the arthritis grade as “Level 1, which is closest to Level 2” may be higher than a probability of determining the arthritis grade as “Level 0 having a greater difference from Level 2 than Level 1” or “Level 3 or Level 4, which is higher than Level 2”.

As described above, this is because the first model 20 has been trained with the training data labeled in the first scheme in which a numerical value of the arthritis grade corresponding to the ground truth value is determined as 1, and a numerical value of the arthritis grade that is closest to and lower than the arthritis grade corresponding to the ground truth value is determined as an arbitrary numerical value that is lower than 1.

In other words, conceptually, the first model 20 may be an artificial intelligence inference model that optimistically determines the arthritis grade (determines the arthritis grade as being better than the actual arthritis grade).

In addition, the second model 30 may have a probability of determining the arthritis grade to be high in the X-ray image (as compared with the first model 20). In detail, the arthritis grade in the second determination information that is output by the second model 30, which has been trained, may be higher than the arthritis grade in the first determination information that is output by the first model 20.

For example, for the X-ray image with the K-L grade of Level 2, the second model 30 may determine with a predetermined probability that the X-ray image is at Level 3, which is higher than Level 2.

Preferably, the second model 30 may determine the arthritis grade as a higher level than the actual arthritis grade of the X-ray image. However, even when the arthritis grade is determined as a higher level than the actual arthritis grade, a probability of determining the arthritis grade as “an arthritis grade that is closest to (with the smallest difference) and higher than the actual arthritis grade” may be higher than a probability of determining the arthritis grade as “an arthritis grade that is not closest to and higher than the actual arthritis grade” or “an arthritis grade that is lower than the actual arthritis grade”.

For example, when the X-ray image with the arthritis grade of Level 2 is input to the second model 30, even when the second model 30 determines the arthritis grade to be higher than Level 2, a probability of determining the arthritis grade as “Level 3, which is closest to Level 2” may be higher than a probability of determining the arthritis grade as “Level 4 having a greater difference from Level 2 than Level 3” or “Level 0 or Level 1, which is lower than Level 2”.

As described above, this is because the second model 30 has been trained with the training data labeled in the second scheme in which a numerical value of the arthritis grade corresponding to the ground truth value is determined as 1, and a numerical value of the arthritis grade that is closest to and higher than the arthritis grade corresponding to the ground truth value is determined as an arbitrary numerical value that is lower than 1.

In other words, conceptually, the second model 30 may be an artificial intelligence inference model that pessimistically determines the arthritis grade (determines the arthritis grade as being worse than the actual arthritis grade).

As described above, the present invention may derive the final determination information for the arthritis grade of the patient by comprehensively using the first determination information and the second determination information output by inputting the identical X-ray image to the first model 20 and the second model 30, which have been trained.

In detail, the present invention may make comprehensive determination on the arthritis grade by considering both an optimistic perspective and a pessimistic perspective while determining the arthritis grade for the X-ray image by using the artificial intelligence inference model.

In addition, according to the present invention, while determining the arthritis grade for the X-ray image by using the artificial intelligence inference model, even when the derived determination information for the arthritis grade is incorrect, a difference from an actual correct arthritis grade may be small. In other words, according to the present invention, even when the determination information for the arthritis grade is output as being incorrect, an incorrect arthritis grade may be output to be similar to actual severity of the arthritis of the patient.

For example, according to the present invention, the final determination information may be preferably output as Level 2 for the X-ray image with the arthritis grade of Level 2. However, even when the final determination information is not output as Level 2, a probability of outputting the final determination information as Level 1 or Level 3, which is close to Level 2, may a probability of outputting the final be greater than determination information as Level 0 or Level 4, which is not close to Level 2.

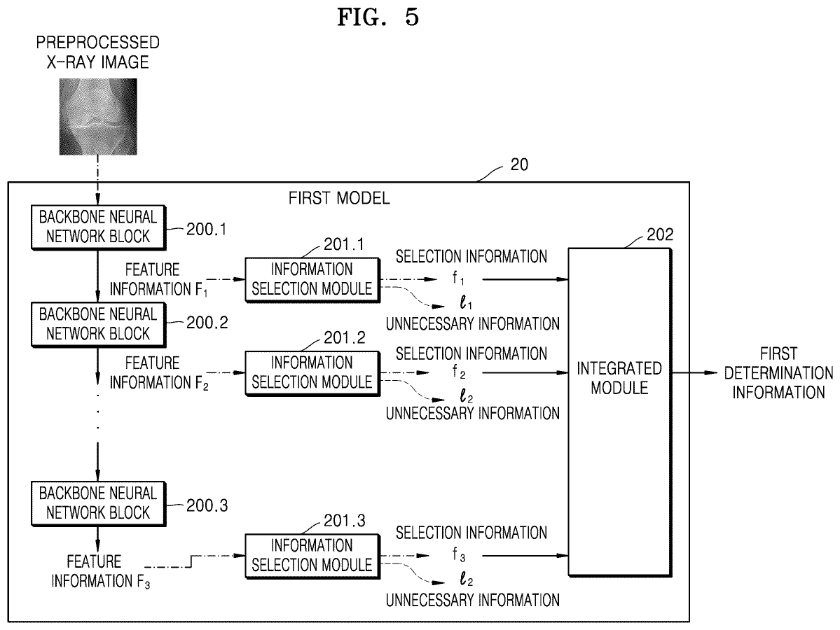

FIG. 5 shows components of the first model 20 according to one embodiment of the present invention.

As shown in FIG. 5, the first model 20 may include: a plurality of deep learning-based backbone neural network blocks for receiving the preprocessed X-ray image or feature information, which is output from another backbone neural network block of a previous stage, so as to output feature information at a corresponding stage; a plurality of deep learning-based information selection modules for receiving the feature information, which is output from the backbone neural network blocks, so as to output selection information associated with the determination of the arthritis grade from the feature information; and an integrated module for receiving information including a plurality of pieces of selection information, which are output from the information selection modules, so as to output the first determination information for the arthritis grade, wherein the backbone neural network blocks and the information selection modules may be artificial neural networks that compress data in an identical scheme.

As described above, according to the present invention, the first model 20 and the second model 30 may be artificial intelligence inference models having substantially the same structure, and only the training data for training the artificial intelligence inference models may be different.

In other words, the second model 30 may also include the components of the first model 20 shown in FIG. 5. Hereinafter, descriptions will be given based on the first model 20.

The first model 20 may include a plurality of deep learning-based backbone neural network blocks for receiving the preprocessed X-ray image or feature information, which is output from another backbone neural network block of a previous stage, so as to output feature information at a corresponding stage.

In detail, the backbone neural network blocks may have a hierarchical structure in which the feature information that is output from the backbone neural network block of the previous stage is input to the backbone neural network block of a subsequent stage.

For example, in FIG. 5, the preprocessed X-ray image may be input to a first backbone neural network block 200.1 so as to output feature information F₁, the output feature information F₁ may be input to a second backbone neural network block 200.2 so as to output feature information F₂, and the output feature information F₂ may be input to a third backbone neural network block 200.3 so as to output feature information F₃.

According to one embodiment of the present invention, the feature information may be data in the form of a feature map expressed by recognizing a shape, a pattern, an edge, and the like in the X-ray image.

The first model 20 may include a plurality of deep learning-based information selection modules for receiving the feature information, which is output from the backbone neural network blocks, so as to output selection information associated with the determination of the arthritis grade from the feature information.

In detail, the information selection module may select data having a specific pattern, a specific edge, or a specific texture from the feature information so as to output the selection information, and the selection information may be data associated with the arthritis grade.

For example, in FIG. 5, the first information selection module 201.1 may distinguish between selection information f₁ associated with the determination of the arthritis grade and unnecessary information l₁ that is not associated with the determination of the arthritis grade in the feature information F₁, the second information selection module 201.2 may distinguish between selection information f₂ associated with the determination of the arthritis grade and unnecessary information l₂ that is not associated with the determination of the arthritis grade in the feature information F₂, and the third information selection module 201.3 may distinguish between selection information f₃ associated with the determination of the arthritis grade and unnecessary information l₃ that is not associated with the determination of the arthritis grade in the feature information F₃.

According to one embodiment of the present invention, the information selection module may be a model trained with training data that is unlabeled or inaccurately labeled so as to select a specific subset (e.g., information associated with arthritis) from input data.

According to one embodiment of the present invention, the number of the information selection modules may correspond to the number of the backbone neural network blocks.

The first model 20 may include an integrated module for receiving information including a plurality of pieces of selection information, which are output from the information selection modules, so as to output the first determination information for the arthritis grade.

In detail, the integrated module may receive a plurality of pieces of selection information f₁, f₂, and f₃, which are output from the information selection modules, respectively, so as to derive the first determination information for the arthritis grade.

According to one embodiment of the present invention, the integrated module may receive at least one of the feature information F₃ that is output from the backbone neural network block of a last stage and the X-ray image that is input to the backbone neural network block of a first stage.

According to one embodiment of the present invention, when the first model 20 is a CNN-based artificial intelligence inference model, the backbone neural network block may include at least one of a convolutional layer for recognizing and extracting a feature from the input data, a pooling layer for reducing or summarizing the feature, and a fully connected layer for calculating a final output.

According to one embodiment of the present invention, when the first model 20 is a transformer-based artificial intelligence inference model, the backbone neural network block may include at least one of a self-attention block for calculating the feature information by considering relation between pieces of input data and a multi-head attention block subjected to parallel training with various patterns.

Meanwhile, the third model 40 and the fourth model 50, which will be described below, may also include a plurality of backbone neural network blocks, a plurality of information selection modules, and an integration module.

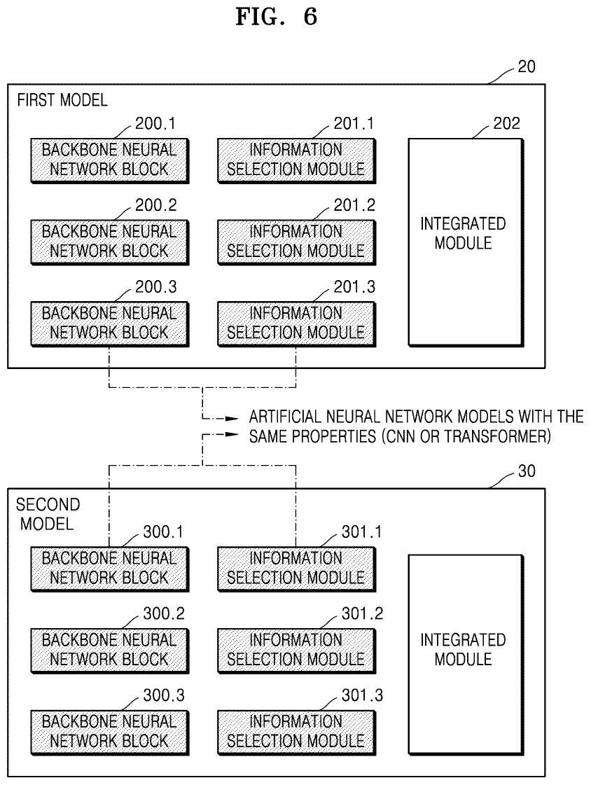

FIG. 6 shows content associated with information processing schemes of the first model 20 and the second model 30 according to one embodiment of the present invention.

As shown in FIG. 6, the backbone neural network blocks and the information selection modules, which constitute the first model 20, may be artificial intelligence inference models having the same properties. Alternatively, the backbone neural network blocks and the information selection modules may be artificial intelligence inference models that process data in an identical scheme.

As described above, the first model 20 may be a CNN-based artificial intelligence inference model or a transformer-based artificial intelligence inference model.

In this case, when the first model 20 is a CNN-based artificial intelligence inference model, all the backbone neural network blocks and the information selection modules, which constitute the first model 20, may be CNN-based artificial intelligence inference models.

Meanwhile, when the first model 20 is a transformer-based artificial intelligence inference model, all the backbone neural network blocks and the information selection modules, which constitute the first model 20, may be transformer-based artificial intelligence inference models.

In addition, as described above, since the second model 30 is substantially the same artificial intelligence inference model as the first model 20, the backbone neural network blocks 200.1, 200.2, 200.3, 300.1, 300.2, and 300.3 and the information selection modules 201.1, 201.2, 201.3, 301.1, 301.2, and 301.3, which constitute the first model 20 and the second model 30, may be artificial intelligence inference models that process data in an identical scheme.

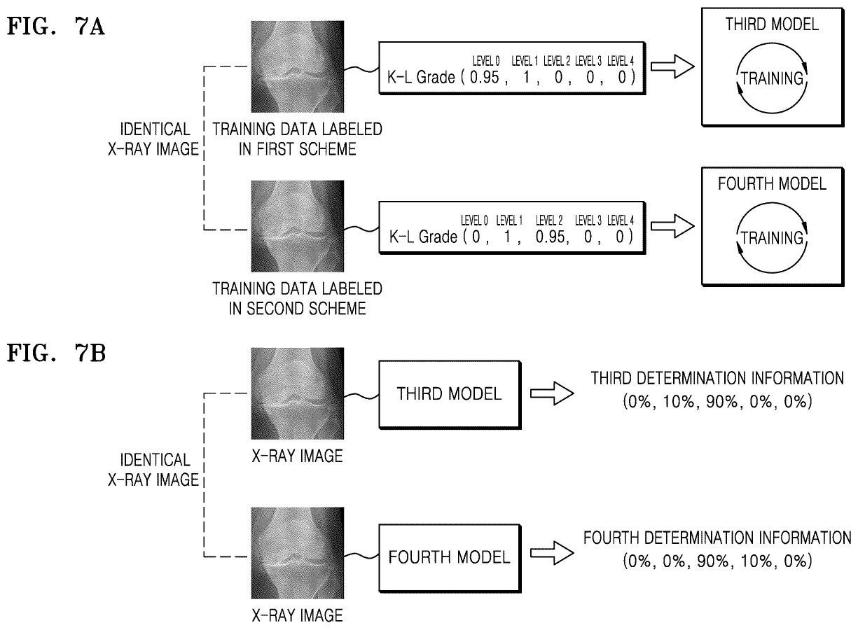

FIGS. 7A and 7B show content associated with training and inference of a third model 40 and a fourth model 50 according to one embodiment of the present invention.

As shown in FIG. 7, the method for determining the arthritis grade may further include: a third determination information derivation step of preprocessing the X-ray image including the joint region, and inputting the preprocessed X-ray image to a third model 40 including an artificial neural network so as to derive third determination information for the arthritis grade; and a fourth determination information derivation step of preprocessing the X-ray image including the joint region, and inputting the preprocessed X-ray image to a fourth model 50 including an artificial neural network so as to derive fourth determination information for the arthritis grade, wherein the comprehensive determination information further includes the third determination information and the fourth determination information, and the third model 40 and the fourth model 50 are artificial neural networks that process or compress data in a different scheme from the first model and the second model.

In addition, the third model 40 may be a deep learning-based artificial neural network model trained by the training data labeled in the first scheme, and the fourth model 50 may be a deep learning-based artificial neural network model trained by the training data labeled in the second scheme.

The third model 40 and the fourth model 50 may be artificial intelligence inference models that determine the arthritis grade for the X-ray image in different data processing schemes from the first model 20 and the second model 30.

In detail, the third model 40 and the fourth model 50 may be transformer-based artificial neural network models when the first model 20 and the second model 30 are CNN-based artificial neural network models. Conversely, the third model 40 and the fourth model 50 may be CNN-based artificial neural network models when the first model 20 and the second model 30 are transformer-based artificial neural network models.

As shown in FIG. 7A, the third model 40 may be trained with the training data labeled in the first scheme (where the labeling information for the arthritis grade is labeled low as compared with the second scheme), and conversely, the fourth model 50 may be trained with the training data labeled in the second scheme (where the labeling information for the arthritis grade is labeled high as compared with the first scheme.

In detail, the third model 40 and the fourth model 50 may be understood as the same artificial intelligence inference models while being trained with different training data for training the artificial intelligence inference model. Preferably, the third model 40 and the fourth model 50 may be understood as being trained with the training data in which only the labeling information is labeled differently for the identical X-ray image.

Preferably, the first model 20 and the second model 30 may be the same artificial intelligence inference models, the third model 40 and the fourth model 50 may be the same artificial intelligence inference models, and the first model 20 and the third model 40 may be artificial intelligence inference models that process data in different schemes.

As described above, the third model 40 and the fourth model 50 may be the same artificial intelligence inference models in which internal structures and data processing schemes may be the same. In detail, when the third model 40 is a CNN-based artificial intelligence inference model, the fourth model 50 may also be a CNN-based artificial intelligence inference model. Meanwhile, when the third model 40 is a transformer-based artificial intelligence inference model, the fourth model 50 may also be a transformer-based artificial intelligence inference model.

As shown in FIG. 7B, the third model 40 may have a high probability of determining the arthritis grade to be low in the X-ray image (as compared with the fourth model 50), and conversely, the fourth model 50 may have a high probability of determining the arthritis grade to be high in the X-ray image (as compared with the third model 40).

In detail, the X-ray image may be input to the third model 40 that has been trained so as to output the third determination information, and the X-ray image may be input to the fourth model 50 that has been trained so as to output the fourth determination information. In this case, a probability that the arthritis grade in the fourth determination information is lower than the arthritis grade in the third determination information may be high.

For example, in FIG. 7B, for the identical X-ray image with the K-L grade of Level 2, the third model 40 may determine with a predetermined probability that the X-ray image is at Level 1, which is lower than Level 2.

Preferably, the third model 40 may determine the arthritis grade as a lower level than an actual arthritis grade of the X-ray image. However, even when the arthritis grade is determined as a lower level than the actual arthritis grade, a probability of determining the arthritis grade as “an arthritis grade that is closest to (with the smallest difference) and lower than the actual arthritis grade” may be higher than a probability of determining the arthritis grade as “an arthritis grade that is not closest to and lower than the actual arthritis grade” or “an arthritis grade that is higher than the actual arthritis grade”.

For example, when the X-ray image with the arthritis grade of Level 2 is input to the third model 40, even when the third model 40 determines the arthritis grade to be lower than Level 2, a probability of determining the arthritis grade as “Level 1, which is closest to Level 2” may be higher than a probability of determining the arthritis grade as “Level 0 having a greater difference from Level 2 than Level 1” or “Level 3 or Level 4, which is higher than Level 2”.

As described above, this is because the third model 40 has been trained with the training data labeled in the first scheme in which a numerical value of the arthritis grade corresponding to the ground truth value is determined as 1, and a numerical value of the arthritis grade that is closest to and lower than the arthritis grade corresponding to the ground truth value is determined as an arbitrary numerical value that is lower than 1.

In other words, conceptually, the third model 40 may be an artificial intelligence inference model that optimistically determines the arthritis grade (determines the arthritis grade as being better than the actual arthritis grade).

In addition, the fourth model 50 may have a probability of determining the arthritis grade to be high in the X-ray image (as compared with the third model 40). In detail, the arthritis grade in the fourth determination information that is output by the fourth model 50, which has been trained, may be higher than the arthritis grade in the third determination information that is output by the third model 40.

For example, for the X-ray image with the K-L grade of Level 2, the fourth model 50 may determine with a predetermined probability that the X-ray image is at Level 3, which is higher than Level 2.

Preferably, the fourth model 50 may determine the arthritis grade as a higher level than the actual arthritis grade of the X-ray image. However, even when the arthritis grade is determined as a higher level than the actual arthritis grade, a probability of determining the arthritis grade as “an arthritis grade that is closest to (with the smallest difference) and higher than the actual arthritis grade” may be higher than a probability of determining the arthritis grade as “an arthritis grade that is not closest to and higher than the actual arthritis grade” or “an arthritis grade that is lower than the actual arthritis grade”.

For example, when the X-ray image with the arthritis grade of Level 2 is input to the fourth model 50, even when the fourth model 50 determines the arthritis grade to be higher than Level 2, a probability of determining the arthritis grade as “Level 3, which is closest to Level 2” may be higher than a probability of determining the arthritis grade as “Level 4 having a greater difference from Level 2 than Level 3” or “Level 0 or Level 1, which is lower than Level 2”.

As described above, this is because the fourth model 50 has been trained with the training data labeled in the second scheme in which a numerical value of the arthritis grade corresponding to the ground truth value is determined as 1, and a numerical value of the arthritis grade that is closest to and higher than the arthritis grade corresponding to the ground truth value is determined as an arbitrary numerical value that is lower than 1.

In other words, conceptually, the fourth model 50 may be an artificial intelligence inference model that pessimistically determines the arthritis grade (determines the arthritis grade as being worse than the actual arthritis grade).

As described above, the present invention may derive the final determination information for the arthritis grade of the patient by comprehensively using the third determination information and the fourth determination information output by inputting the identical X-ray image to the third model 40 and the fourth model 50, which have been trained.

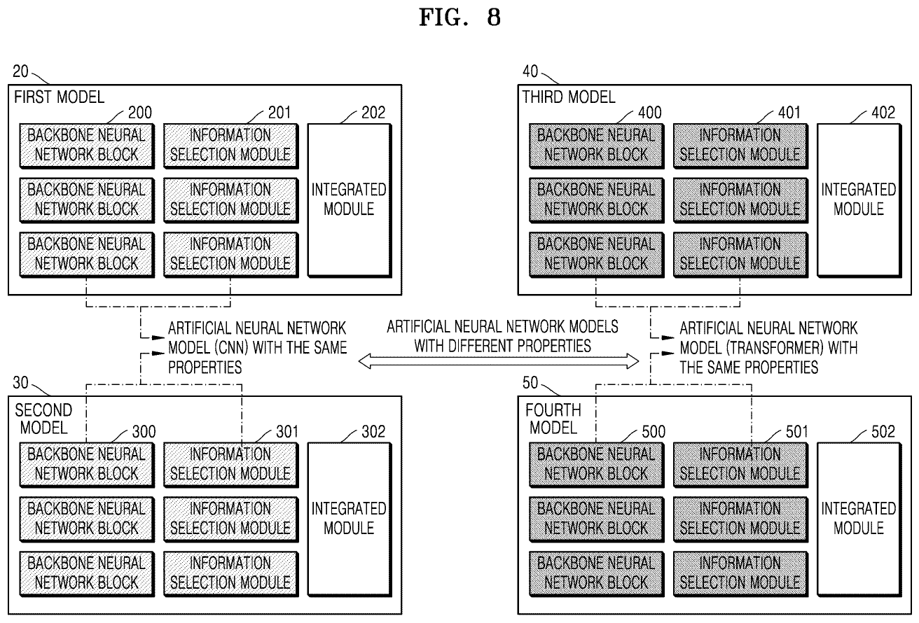

FIG. 8 shows content associated with information processing schemes of the third model 40 and the fourth model 50 according to one embodiment of the present invention.

As shown in FIG. 8, the first model 20 and the second model 30 may be convolutional neural network (CNN)- or transformer-based artificial neural network models, and the third model 40 and the fourth model 50 may be: transformer-based artificial neural network models when the first model 20 and the second model 30 are CNN-based artificial neural network models; and CNN-based artificial neural network models when the first model 20 and the second model 30 are transformer-based artificial neural network models.

In detail, the third model 40 and the fourth model 50 may also include a plurality of backbone neural network blocks 400 and 500, a plurality of information selection modules 401 and 501, and integration modules 402 and 502, and an operation of each of the components is substantially identical to the operation in FIG. 5, so that redundant descriptions thereof will be omitted.

In addition, the backbone neural network blocks 400 and 500 and the information selection modules 401 and 501, which in constitute the third model 40 and the fourth model 50, may process data in an identical scheme. For example, when the third model 40 and the fourth model 50 are CNN-based artificial intelligence inference models, all the backbone neural network blocks 400 and 500 and the information selection modules 401 and 501, which constitute the third model 40 and the fourth model 50, may be CNN-based artificial intelligence inference models.

Meanwhile, when the third model 40 and the fourth model 50 are transformer-based artificial intelligence inference models, all the backbone neural network blocks 400 and 500 and the information selection modules 401 and 501, which constitute the third model 40 and the fourth model 50, may be transformer-based artificial intelligence inference models.

As a result, the backbone neural network blocks 200 and 300 and the information selection modules 201 and 301, which constitute the first model 20 and the second model 30, may be artificial neural network blocks that process data in a CNN- or transformer-based scheme, and the backbone neural network blocks 400 and 500 and the information selection modules 401 and 501, which constitute the third model 40 and the fourth model 50, may be artificial neural network blocks that process data in a CNN- or transformer-based scheme (in a scheme that is different from the scheme of the first model 20 and the second model 30).

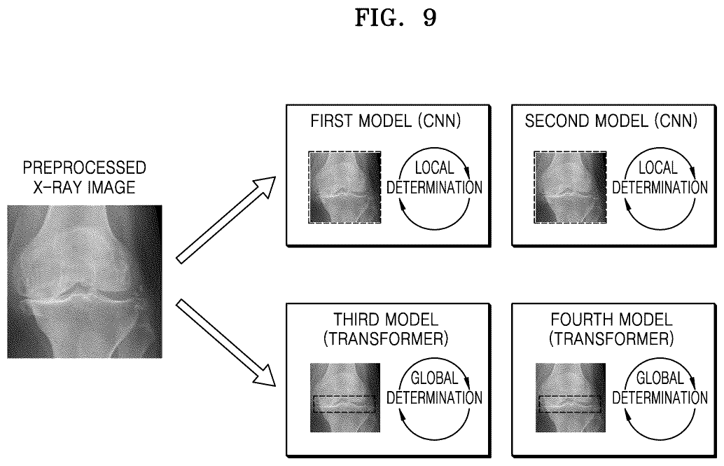

FIG. 9 shows content about determination schemes of the first model 20 and the third model 40 according to one embodiment of the present invention.

Hereinafter, descriptions will be given while assuming that the first model 20 and the second model 30 are CNN-based artificial intelligence inference models, and the third model 40 and the fourth model 50 are transformer-based artificial intelligence inference models.

According to one embodiment of the present invention, a CNN-based artificial intelligence inference model may locally analyze a specific portion of an input X-ray image so as to derive determination information for the arthritis grade. In detail, the CNN-based artificial intelligence inference model may operate in a scheme of dividing the image into regions having small sizes and analyzing characteristics of each of the regions, thereby analyzing a local pattern of the specific portion associated with the arthritis in the X-ray image so as to derive the determination information.

Meanwhile, a transformer-based artificial intelligence inference model may globally analyze the input X-ray image so as to derive the determination information for the arthritis grade. In detail, the transformer-based artificial intelligence inference model may operate in a scheme of analyzing globally the image with a self-attention algorithm, thereby analyzing a global pattern of the X-ray image so as to derive the determination information.

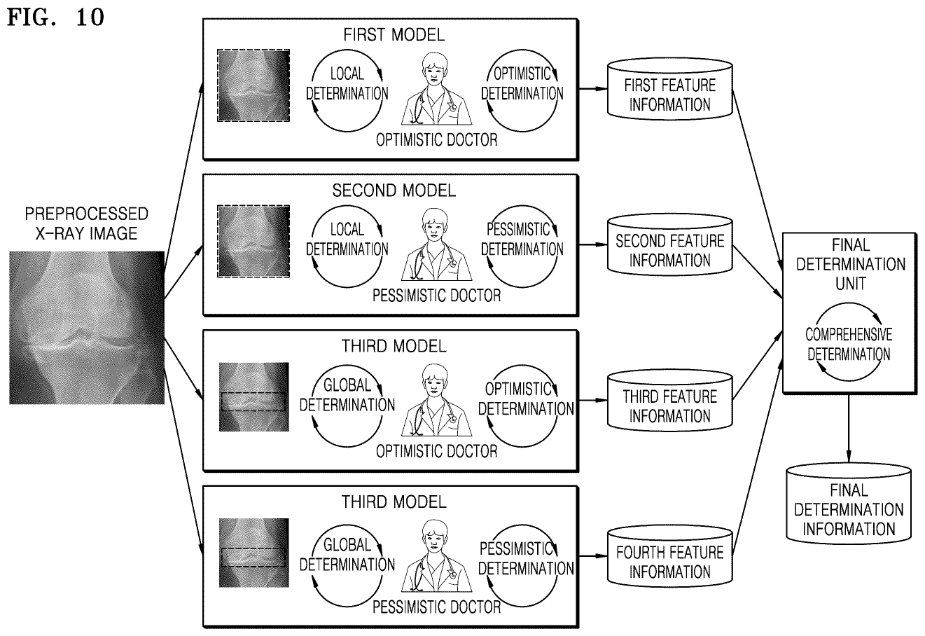

FIG. 10 shows content about a method for determining an arthritis grade according to one embodiment of the present invention.

As shown in FIG. 10, the first model 20, which is a CNN-based artificial intelligence inference model trained with the training data labeled in the first scheme, may be a model that locally determines the X-ray image in an optimistic perspective. In other words, the first model 20 may be understood as representing a doctor who locally analyzes the X-ray image in the optimistic perspective in an actual medical site.

In addition, the second model 30, which is a CNN-based artificial intelligence inference model trained with the training data labeled in the second scheme, may be a model that locally determines the X-ray image in a pessimistic perspective. In other words, the second model 30 may be understood as representing a doctor who locally analyzes the X-ray image in the pessimistic perspective in the actual medical site.

In addition, the third model 40, which is a transformer-based artificial intelligence inference model trained with the training data labeled in the first scheme, may be a model that determines the X-ray image in the optimistic globally perspective. In other words, the third model 40 may be understood as representing a doctor who globally analyzes the X-ray image in the optimistic perspective in the actual medical site.

In addition, the fourth model 50, which is a transformer-based artificial intelligence inference model trained with the training data labeled in the second scheme, may be a model that globally determines the X-ray image in the pessimistic perspective. In other words, the fourth model 50 may be understood as representing a doctor who globally analyzes the X-ray image in the pessimistic perspective in the actual medical site.

According to one embodiment of the present invention, a process that is similar to a process in which the medical staff determines the arthritis grade of the patient by locally or globally determining the X-ray image in the pessimistic or optimistic perspective in the actual medical site may be implemented.

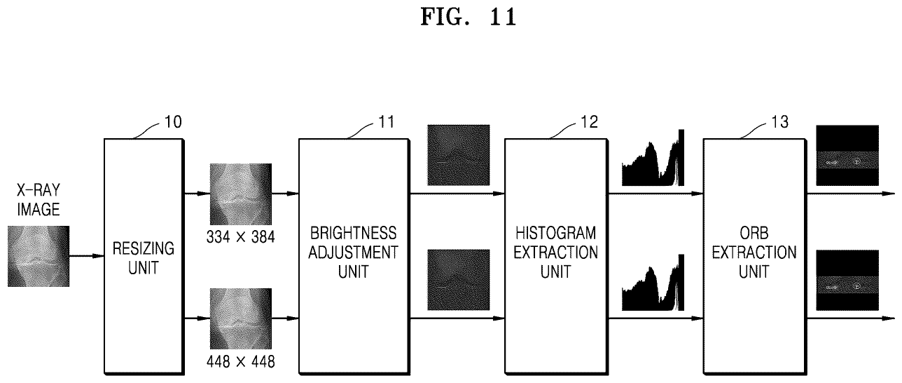

FIG. 11 shows a preprocessing process according to one embodiment of the present invention.

As shown in FIG. 11, the X-ray image may be preprocessed and input to an artificial intelligence inference model (the first model 20, the second model 30, the third model 40, and the fourth model 50), and the preprocessing process may include at least one of resizing, brightness adjustment, histogram extraction, and ORB extraction.

In detail, the resizing unit 10 may process a size of the X-ray image into a form that is suitable for the artificial intelligence inference model. For example, the resizing unit 10 may process the X-ray image into a size that is suitable for each of the CNN-based artificial intelligence inference model and the transformer-based artificial intelligence inference model.

The brightness adjustment unit 11 may process brightness of the X-ray image into a form that is suitable for the artificial intelligence inference model.

The histogram extraction unit 12 may analyze the brightness in the X-ray image so as to extract a histogram having brightness distribution of 0 to 255.

The ORB extraction unit 13 may extract ORB information by recognizing a feature point in the X-ray image. In detail, the ORB extraction unit 13 may extract ORB information for the X-ray image by using a features-from-accelerated-segment-test (FAST) algorithm that detects a local feature point such as an edge of the image and a binary-robust-independent-elementary-features (BRIEF) algorithm that converts a patch around a recognized feature point into a binary vector.

As described above, the X-ray image with an adjusted size and adjusted brightness or the histogram and ORB the information extracted from the X-ray image may be input to the artificial intelligence inference model.

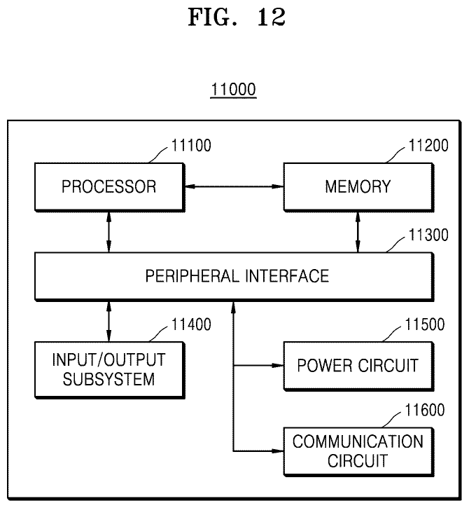

FIG. 12 schematically shows internal components of the computing device according to one embodiment of the present invention.

System for determining an arthritis grade shown in the above-described FIG. 2 may include components of the computing device 11000 shown in FIG. 12.

As shown in FIG. 12, the computing device 11000 may at least include at least one processor 11100, a memory 11200, a peripheral device interface 11300, an input/output subsystem (I/O subsystem) 11400, a power circuit 11500, and a communication circuit 11600. The computing device 11000 may correspond to the system for determining an arthritis grade shown in FIG. 2.

The memory 11200 may include, for example, a high-speed random access memory, a magnetic disk, an SRAM, a DRAM, a ROM, a flash memory, or a non-volatile memory. The memory 11200 may include a software module, an instruction set, or other various data necessary for the operation of the computing device 11000.

The access to the memory 11200 from other components of the processor 11100 or the peripheral interface 11300, may be controlled by the processor 11100.

The peripheral interface 11300 may combine an input and/or output peripheral device of the computing device 11000 to the processor 11100 and the memory 11200. The processor 11100 may execute the software module or the instruction set stored in memory 11200, thereby performing various functions for the computing device 11000 and processing data.

The input/output subsystem may combine various input/output peripheral devices to the peripheral interface 11300. For example, the input/output subsystem may include a controller for combining the peripheral device such as monitor, keyboard, mouse, printer, or a touch screen or sensor, if needed, to the peripheral interface 11300. According to another aspect, the input/output peripheral devices may be combined to the peripheral interface 11300 without passing through the I/O subsystem.

The power circuit 11500 may provide power to all or a portion of the components of the terminal. For example, the power circuit 11500 may include a power failure detection circuit, a power converter or inverter, a power status indicator, a power failure detection circuit, a power converter or inverter, a power status indicator, or any other components for generating, managing, and distributing the power.

The communication circuit 11600 may use at least one external port, thereby enabling communication with other computing devices.

Alternatively, as described above, if necessary, the communication circuit 11600 may transmit and receive an RF signal, also known as an electromagnetic signal, including RF circuitry, thereby enabling communication with other computing devices.

The above embodiment of FIG. 12 is merely an example of the computing device 11000, and the computing device 11000 may have a configuration or arrangement in which some components shown in FIG. 12 are omitted, additional components not shown in FIG. 12 are further provided, or at least two components are combined. For example, a computing device for a communication terminal in a mobile environment may further include a touch screen, a sensor or the like in addition to the components shown in FIG. 12, and the communication circuit 11600 may include a circuit for RF communication of various communication schemes (such as WiFi, 3G, LTE, Bluetooth, NFC, and Zigbee). The components that may be included in the computing device 11000 may be implemented by hardware, software, or a combination of both hardware and software which include at least one integrated circuit specialized in a signal processing or an application.

The methods according to the embodiments of the present invention may be implemented in the form of program instructions to be executed through various computing devices, thereby being recorded in a computer-readable medium. In particular, a program according to an embodiment of the present invention may be configured as a PC-based program or an application dedicated to a mobile terminal. The application to which the present invention is applied may be installed in the computing device 11000 through a file provided by a file distribution system. For example, a file distribution system may include a file transmission unit (not shown) that transmits the file according to the request of the computing device 11000.

The above-mentioned device may be implemented by hardware components, software components, and/or a combination of hardware components and software components. For example, the devices and components described in the embodiments may be implemented by using at least one general purpose computer or special purpose computer, such as a processor, a controller, an arithmetic logic unit (ALU), a digital signal processor, a microcomputer, a field programmable gate array (FPGA), a programmable logic unit (PLU), a microprocessor, or any other device capable of executing and responding to instructions. The processing device may execute an operating system (OS) and at least one software application executed on the operating system. In addition, the processing device may access, store, manipulate, process, and create data in response to the execution of the software. For the further understanding, some cases may have described that one processing device is used, however, it is well known by those skilled in the art that the processing device may include a plurality of processing elements and/or a plurality of types of processing elements. For example, the processing device may include a plurality of processors or one processor and one controller. In addition, other processing configurations, such as a parallel processor, are also possible.

The software may include a computer program, a code, and an instruction, or a combination of at least one thereof, and may configure the processing device to operate as desired, or may instruct the processing device independently or collectively. In order to be interpreted by the processor or to provide instructions or data to the processor, the software and/or data may be permanently or temporarily embodied in any type of machine, component, physical device, virtual equipment, computer storage medium or device, or in a signal wave to be transmitted. The software may be distributed over computing devices connected to networks, so as to be stored or executed in a distributed manner. The software and data may be stored in at least one computer-readable recording medium.

The method according to the embodiment may be implemented in the form of program instructions to be executed through various computing mechanisms, thereby being recorded in a computer-readable medium. The computer-readable medium may include program instructions, data files, data structures, and the like, independently or in combination thereof. The program instructions recorded on the medium may be specially designed and configured for the embodiment, or may be known to those skilled in the art of computer software so as to be used. An example of the computer-readable medium includes a magnetic medium such as a hard disk, a floppy disk and a magnetic tape, an optical medium such as a CD-ROM and a DVD, a magneto-optical medium such as a floptical disk, and a hardware device specially configured to store and execute a program instruction such as ROM, RAM, and flash memory. An example of the program instruction includes a high-level language code to be executed by a computer using an interpreter or the like as well as a machine code generated by a compiler. The above hardware device may be configured to operate as at least one software module to perform the operations of the embodiments, and vise versa.

Although the above embodiments have been described with reference to the limited embodiments and drawings, however, it will be understood by those skilled in the art that various changes and modifications may be made from the above-mentioned description. For example, even though the described descriptions may be performed in an order different from the described manner, and/or the described components such as system, structure, device, and circuit may be coupled or combined in a form different from the described manner, or replaced or substituted by other components or equivalents, appropriate results may be achieved.

Therefore, other implementations, other embodiments, and equivalents to the claims are also within the scope of the following claims.

What is claimed is:

1. A method for determining an arthritis grade, which is performed by a computing system including at least one processor and at least one memory, the method comprising:
a first determination information derivation step of preprocessing an X-ray image including a joint region, and inputting the preprocessed X-ray image to a first model including an artificial neural network so as to derive first determination information for the arthritis grade;
a second determination information derivation step of preprocessing the X-ray image including the joint region, and inputting the preprocessed X-ray image to a second model including an artificial neural network so as to derive second determination information for the arthritis grade; and
a final determination step of deriving final determination information for the arthritis grade based on comprehensive determination information including the first determination information and the second determination information,
wherein the first model is a deep learning-based artificial neural network model trained by training data labeled in a first scheme,
the second model is a deep learning-based artificial neural network model trained by the training data labeled in a second scheme,
the first scheme labels labeling information for the arthritis grade to be low as compared with the second scheme for identical training data, which is the X-ray image, and
the arthritis grade is numerically expressed in proportion to or in inverse proportion to severity of arthritis.

2. The method of claim 1, wherein the labeling information for the training data before being labeled in the first scheme or the second scheme is determined as a one-hot vector in which the arthritis grade corresponding to a ground truth value of the training data is determined as 1, and the arthritis grade that does not correspond to the ground truth value is determined as 0 in the arthritis grade divided in levels,
the first scheme is a scheme of inputting an arbitrary number that is lower than 1 in the arthritis grade that is lower than the arthritis grade corresponding to 1 in the labeling information in a form of a one-hot vector, and
the second scheme is a scheme of inputting an arbitrary number that is lower than 1 in the arthritis grade that is higher than the arthritis grade corresponding to 1 in the labeling information of the training data.

3. The method of claim 1, wherein the first model is trained such that a probability of determining the arthritis grade to be lower than the arthritis grade corresponding to a ground truth value of the training data occurs, and
the second model is trained such that a probability of determining the arthritis grade to be higher than the arthritis grade corresponding to the ground truth value of the training data occurs.

4. The method of claim 1, wherein the first model and the second model are trained with the training data, which is an identical X-ray image, to which the labeling information is assigned in the first scheme or the second scheme so that only a labeling scheme is different, and
the first determination information and the second determination information include information associated with a numerical value representing that the input preprocessed X-ray image is predicted to correspond to each of a plurality of arthritis grades.

5. The method of claim 1, wherein the first model includes:
a plurality of deep learning-based backbone neural network blocks for receiving the preprocessed X-ray image or feature information, which is output from another backbone neural network block of a previous stage, so as to output feature information at a corresponding stage;
a plurality of deep learning-based information selection modules for receiving the feature information, which is output from the backbone neural network blocks, so as to output selection information associated with the determination of the arthritis grade from the feature information; and
an integrated module for receiving information including a plurality of pieces of selection information, which are output from the information selection modules, so as to output the first determination information for the arthritis grade, and
the backbone neural network blocks and the information selection modules are artificial neural networks that compress data in an identical scheme.

6. The method of claim 1, further comprising:
a third determination information derivation step of preprocessing the X-ray image including the joint region, and inputting the preprocessed X-ray image to a third model including an artificial neural network so as to derive third determination information for the arthritis grade; and
a fourth determination information derivation step of preprocessing the X-ray image including the joint region, and inputting the preprocessed X-ray image to a fourth model including an artificial neural network so as to derive fourth determination information for the arthritis grade,
wherein the comprehensive determination information further includes the third determination information and the fourth determination information, and
the third model and the fourth model are artificial neural networks that process or compress data in a different scheme from the first model and the second model.

7. The method of claim 6, wherein the first model and the second model are convolutional neural network (CNN)- or transformer-based artificial neural network models, and
the third model and the fourth model are:
transformer-based artificial neural network models when the first model and the second model are CNN-based artificial neural network models; and
CNN-based artificial neural network models when the first model and the second model are transformer-based artificial neural network models.

8. The method of claim 6, wherein the third model is a deep learning-based artificial neural network model trained by the training data labeled in the first scheme, and
the fourth model is a deep learning-based artificial neural network model trained by the training data labeled in the second scheme.

9. A system for determining an arthritis grade, which includes at least one processor and at least one memory, the system comprising:
a first determination information derivation unit for preprocessing an X-ray image including a joint region, and inputting the preprocessed X-ray image to a first model including an artificial neural network so as to derive first determination information for the arthritis grade;
a second determination information derivation unit for preprocessing the X-ray image including the joint region, and inputting the preprocessed X-ray image to a second model including an artificial neural network so as to derive second determination information for the arthritis grade; and
a final determination unit for deriving final determination information for the arthritis grade based on comprehensive determination information including the first determination information and the second determination information,
wherein the first model is a deep learning-based artificial neural network model trained by training data labeled in a first scheme,
the second model is a deep learning-based artificial neural network model trained by the training data labeled in a second scheme,
the first scheme labels labeling information for the arthritis grade to be low as compared with the second scheme for identical training data, which is the X-ray image, and
the arthritis grade is numerically expressed in proportion to or in inverse proportion to severity of arthritis.

10. A computer-readable recording medium including at least one processor and at least one memory, and configured to perform a method for determining an arthritis grade, wherein the computer-readable recording medium stores instructions for performing steps including:
a first determination information derivation step of preprocessing an X-ray image including a joint region, and inputting the preprocessed X-ray image to a first model including an artificial neural network so as to derive first determination information for the arthritis grade;
a second determination information derivation step of preprocessing the X-ray image including the joint region, and inputting the preprocessed X-ray image to a second model including an artificial neural network so as to derive second determination information for the arthritis grade; and
a final determination step of deriving final determination information for the arthritis grade based on comprehensive determination information including the first determination information and the second determination information,
the first model is a deep learning-based artificial neural network model trained by training data labeled in a first scheme,
the second model is a deep learning-based artificial neural network model trained by the training data labeled in a second scheme,
the first scheme labels labeling information for the arthritis grade to be low as compared with the second scheme for identical training data, which is the X-ray image, and
the arthritis grade is numerically expressed in proportion to or in inverse proportion to severity of arthritis.
